(19) United States
(12) Patent Application Publication
(10) Pub. No.: US 2012/0114606 A1
(43) Pub. Date: May 10, 2012

(54) Novel Pasteuria Strain and Uses Thereof

(76) Inventors: Thomas E. HEWLETT, High Springs, FL (US); Liesbeth M. SCHMIDT, Lake City, FL (US); April GREEN, Gainesville, FL (US); Charles S. BARMORE, Gainesville, FL (US)

(21) Appl. No.: 13/284,427

(22) Filed: Oct. 28, 2011

Related U.S. Application Data
(60) Provisional application No. 61/411,613, filed on Nov. 9, 2010.

(51) Int. Cl. A01N 63/02; A01P 5/00; C12N 1/20; C12N 1/21

(52) U.S. Cl. 424/93.2; 435/252.3; 435/252.1; 424/93.4

(57) ABSTRACT

The subject invention provides a novel and advantageous strain of Pasteuria bacteria with nematicidal activity against lance nematodes. The subject invention provides the novel bacterial culture referred to as ATCC SD-5832, and mutants or variants thereof. Also provided are nematicidal compositions comprising the Pasteuria strain or its mutants or variants, and uses thereof for treating phytopathogenic and soil-dwelling nematodes.

### CROSS-REFERENCE TO A RELATED APPLICATION

This application claims the benefit of U.S. Provisional Application Ser. No. 61/411,613, filed Nov. 9, 2010, the disclosure of which is hereby incorporated by reference in its entirety, including all figures, tables or drawings.

### BACKGROUND OF INVENTION

Worldwide crop losses caused by plant parasitic nematodes exceed $100 billion annually. Preventing this damage represents a significant challenge. Meanwhile, the use and production of one of the most extensively used nematicides, fumigant methyl bromide, have been significantly curtailed by the Montreal Protocol due to the ozone depleting effects of methyl bromide. At present, there is insufficient time to develop and register new synthetic compounds for nematode control. Thus, materials and methods that effectively control nematodes are urgently needed.

Phytopathogenic nematodes are particularly difficult to control because they are covered with a thick, impermeable cuticle, or outer covering, and have very few sensory neurons. Since many pest control compounds operate as neurotoxins, the low number of neurons exposed by phytopathogenic nematodes decreases the effective target area for nematicidal compounds and has resulted in the development of nematicidal compounds with exquisitely high neurotoxic properties.

Furthermore, because phytopathogenic nematodes are found in soil or plant roots, exposing the phytopathogenic nematodes to control agents is difficult to achieve and puts the water table at risk of contamination from those toxic compounds. The use of nematicides based on neurotoxins contaminates both ground and surface water. Consequently, many of these compounds are being removed from the market for public health reasons.

The lance nematode, also known as Hoplolaimus galeatus, is the most economically damaging nematode to turf grasses. Lance nematodes cause extensive damage in the root system of plants by embedding the anterior end, or sometimes the entire body, inside roots. They not only feed on the roots, but also create wounds on the root system, and thus cause the plants to be more susceptible to other disease-causing organisms.

Lance nematodes parasitize the roots of a wide variety of plant species, including turf grasses, cotton, cowpea, sweet potato, soybean, pineapple, tea, peanut, wheat, rice, sugarcane, sorghum, tobacco, and various vegetables such as tomato, okra, squash, and lettuce. Pathogenicity of lance nematodes has greatly impacted agriculture and landscaping. For example, they cause excessive damages to turf grasses along the East Coast of the United States from New England to Florida and the Mississippi River basin, and internationally, in Canada, India, Tanzania, and Central and South America.

Fumigation of soil prior to planting is a popular method for controlling nematodes. One of the most popular fumigants, methyl bromide, is slated for removal from use because of its ozone destroying properties. Furthermore, this practice of soil fumigation kills organisms in soil indiscriminately and runs the risk of eliminating beneficial microbes. The overall market for an effective nematicide with benign environmental effects is estimated to approach one billion dollars on a world-wide basis.

Pasteuria was first described in 1888 by Metchnikoff (Annales de I'Institut Pasteur 2:165-170) as a parasite of water fleas. Subsequently, Cobb described a Pasteuria infection of the nematode Dorylaimus bulbiferous (2^{nd} ed. Hawaiian Sugar Planters Assoc., Expt. Sta. Div. Path. Physiol. Bull. 5:163-195, 1906).

The life cycle of the bacteria begins when endospores bind to the cuticle of the nematodes in soil. Pasteuria proliferate within the nematode body and pass through several documented morphological phases, including mycelial structures and thalli, culminating in the development of endospores. Endospores are released when the nematode body lyses.

Growth of the bacteria within the nematode body reduces or eliminates the production of eggs by the nematode, severely restricting the rate of nematode reproduction. Economic damage to the host crop normally is inflicted by the first generation progeny of nematodes and is prevented by Pasteuria through lowering the concentration of progeny nematodes in the plant root zone.

While Pasteuria strains have been produced on multiple nematode species, such as Meloidogyne incognita (Verdeho, S, and R. Mankau. 1986. Journal of Nematology, 18:635) and Meloidogyne arenaria (U.S. Pat. No. 6,919,197), no Pasteuria strain has been observed or successfully cultivated on lance nematodes prior to now.

### BRIEF SUMMARY

The subject invention provides a new and advantageous strain of Pasteuria bacteria that parasitizes lance nematodes. This strain has been deposited with the American Type Culture Collection and has been assigned the deposit number ATCC SD-5832. These bacteria are able to produce endospores that have the unique and useful property of being able to attach to, infect, grow in, re-sporulate in, and kill lance nematodes and other phytopathogenic nematodes.

The subject invention also encompasses mutants of the disclosed Pasteuria strain that have substantially the same or improved nematicidal properties. Procedures for making mutants are well known in the microbiological art. For example, ultraviolet light and nitrosoguanidine are used extensively toward this end.

The subject invention further pertains to variants of the exemplified microbes. The variants can be identified by, for example, polynucleotide sequences that are highly homologous with sequences from the exemplified isolate as well as by having the desired biological activity against lance nematodes.

The subject invention further includes compositions comprising a nematicidally effective amount of endospores of the disclosed Pasteuria bacterial strain and the use of these compositions to control phytopathogenic nematodes.

In one embodiment, a plant seed is first treated with an adherent that can adhere to the Pasteuria spores and/or a composition containing the spores. The adherent can be, for example, a glue and/or one or more polymers or copolymers. Examples of adherents include, but are not limited to, glues (such as ELMERS™ glue); polyvinyl acetates; silicone materials; and natural inorganic materials such as silica gel and clay.

Another aspect of the subject invention provides a seed having at least part of its surface coated with a Pasteuria composition, wherein the Pasteuria composition comprises an effective amount of Pasteuria spores for nematode control.

### DETAILED DISCLOSURE

The novel bacterial strain of the subject invention has nematicidal activity against phytopathogenic nematodes including lance nematodes (Hoplolaimus galeatus). A culture of the microbe has been deposited with the American Type Culture Collection (ATCC), 10801 University Blvd., Manassas, Va. 20110-2209 USA. The deposit has been assigned accession number ATCC No. SD-5832 by the repository and was deposited on Jan. 13, 2010.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

As used herein, reference to “isolated” means that the strain is removed from the environment in which it exists in nature. Thus, the isolated strain may exist as, for example, a biologically pure culture, or as spores (or other forms of the strain) in association with an agricultural carrier.

As used herein, the term “comprising” further contemplates scenarios in which the composition and/or method “consists of” or “consists essentially of” the recited components and/or steps. As used herein, reference to “consists essentially of” refers to the situation where additional components and/or steps are only those that do not affect the pesticidal activity of the composition and/or method.

“A nematicidally effective amount,” as used herein, refers to an amount of Pasteuria spores capable of killing, controlling, or infecting nematodes; retarding the growth or reproduction of nematodes; reducing a nematode population; and/or reducing damage to plants caused by nematodes.

In specific embodiments, the subject invention provides bacterial strain ATCC SD-5832 and mutants thereof. Procedures for making mutants are well known in the microbiological art. For example, ultraviolet light and nitrosoguanidine are used extensively toward this end. In other aspects, the invention provides variants of ATCC SD-5832 having nematicidal activity.

In one embodiment a “variant” includes a strain that has a polynucleotide sequence that hybridizes under high stringency conditions with the entire sequence, or a fragment thereof with at least 100 nucleic acids, of any of the followings sequences: 16S rDNA sequence, F1-Atpase sequence, spoIIAB sequence, an ATP synthase subunit sequence such as ATP synthase b subunit sequence, atpF sequence, or atpA sequence of ATCC SD-5832.

“Hybridization” refers to a reaction in which one or more polynucleotides react to form a complex that is stabilized via hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. Various degrees of stringency of hybridization can be employed. The more severe the conditions, the greater the complementarity that is required for duplex formation. Severity of conditions can be controlled by temperature, probe concentration, probe length, ionic strength, time, and the like.

Preferably, hybridization is conducted under high stringency conditions by techniques well known in the art, as described, for example, in Keller, G. H. & M. M. Manak, DNA Probes, and the companion volume DNA Probes: Background, Applications, Procedures (various editions, including 2^{nd} Edition, Nature Publishing Group, 1993). Hybridization is also described extensively in the Molecular Cloning manuals published by Cold Spring Harbor Laboratory Press, including Sambrook & Russell, Molecular Cloning: A Laboratory Manual (2001). Each of these publications is incorporated herein by reference in its entirety.

A non-limiting example of high stringency conditions for hybridization is at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C. A non-limiting example of hybridization conditions are conditions selected to be about 1, 2, 3, 4, 5, 6, 7, 8, 9, 10, 11, 12, 13, 14, 15, 16, 17, 18, 19, 20, 21, 22, 23, 24, or 25° C. lower than the thermal melting point (Tₘ) for the specific sequence in the particular solution. Tₘ is the temperature (dependent upon ionic strength and pH) at which 50% of the target sequence hybridizes to a perfectly matched probe. Tₘ typically increases with [Na⁺] concentration because the sodium cations electrostatically shield the anionic phosphate groups of the nucleotides and minimize their repulsion. The washes employed may be for about 5, 10, 15, 20, 25, 30, or more minutes each, and may be of increasing stringency if desired.

Calculations for estimating Tₘ are well-known in the art. For example, the melting temperature may be described by the following formula (Beltz, G. A., K. A. Jacobs, T. H. Eickbush, P. T. Cherbas, and F. C. Kafatos, Methods of Enzymology, R. Wu, L. Grossman and K. Moldave [eds.] Academic Press, New York 100:266-285, 1983).

Tm=81.5° C.+16.6 Log [Na+]+0.41(% G+C)−0.61(% formamide)−600/length of duplex in base pairs.

A more accurate estimation of Tₘ may be obtained using nearest-neighbor models. Breslauer, et al., Proc. Natl. Acad. Sci. USA, 83:3746-3750 (1986); SantaLucia, Proc. Natl. Acad. Sci. USA, 95: 1460-1465 (1998); Allawi & SantaLucia, Biochemistry 36:10581-94 (1997); Sugimoto et al., Nucleic Acids Res., 24:4501-4505 (1996). Tₘ may also be routinely measured by differential scanning calorimetry (Duguid et al., Biophys J, 71:3350-60, 1996) in a chosen solution, or by other methods known in the art, such as UV-monitored melting. As the stringency of the hydridization conditions is increased, higher degrees of homology are obtained.

Typical methods that can be used to identify the presence of the DNA sequence as described herein, include and are not limited to, detecting a specific DNA sequence hybridization using specific oligonucleotides, direct DNA sequencing, restriction enzyme digest. RNase protection, chemical cleavage, and ligase-mediated detection.

Alternatively or additionally, an example of a variant of ATCC SD-5832 is a strain containing a polynucleotide that has greater than about 90%, 95%, 96%, 97%, 98%, 98.5%, 99%, or 99.5% sequence identity to the entire sequence or a fragment thereof of any of the followings sequences: 16S rDNA sequence, F1-Atpase sequence, spoIIAB sequence, an ATP synthase subunit sequence such as ATP synthase b subunit sequence, atpF sequence, or atpA sequence of ATCC SD-5832, wherein the variant has nematicidal activity.

The crystal structure of an F1-Atpase is given by Stocker et al., Structure 15(8):904-914 (2007) and the function of F1-Atpase has been extensively studied. See, e.g., Itoh et al., “Mechanically driven ATP synthesis by F1-Atpase,” Nature 427(6973):407-8 (2004), as well as the references cited therein. In certain embodiments of the invention, the variant sequences of atpA encode a polypeptide that retains at least about 90%, 95%, 96%, 97%, 98%, 98.5%, 99%, or 99.5% sequence identity to the entire sequence of F1-Atpase of ATCC SD-5832, or a fragment thereof, wherein the variant has nematicidal activity.

The spoIIAB protein is an anti-sigma factor. Duncan & Losick, Proc Natl Acad Sci USA, 90(6): 2325-2329 (1993). A variety of crystal structures are available. Masuda et al., J Mol Biol, 340(5):941-956 (2004); Campbell et al., Cell, 108(6):795-807 (2002). In certain embodiments of the invention, the variant sequences of spoIIAB encode a polypeptide that retains at least about 90%, 95%, 96%, 97%, 98%, 98.5%, 99%, or 99.5% sequence identity to the entire sequence of spoIIAB of ATCC SD-5832, or a fragment thereof, wherein the variant has nematicidal activity.

Structural and functional data for E. coli ATP synthase b subunit is given, for example, by Del Rizzo et al., J Mol Biol, 364(4):735-46 (2006); and Claggett et al., J Bacteriol, 189(15):5463-5471 (207). In certain embodiments of the invention, the variant sequences of atpF encode a polypeptide that retains at least about 90%, 95%, 96%, 97%, 98%, 98.5%, 99%, 99.5% sequence identity to the entire sequence of atpF of ATCC SD-5832, or a fragment thereof, wherein the variant has nematicidal activity.

In certain embodiments, strains of the present invention that parasitize lance nematodes are those Pasteuria strains that are phylogenetically more closely related to ATCC SD-5832 than to any currently known Pasteuria strain (or, alternatively, more closely related to ATCC SD-5832 than to any known non-lance-parasitizing Pasteuria penetrans strain), as determined by routine analysis of 16s ribosomal sequences.

A variety of tools and data suitable for analysis of 16s rDNA are known in the art. The following accession numbers returned by NCBI Blast of database “nr” provide 16s ribosomal sequences referenced by NCBI gi number: 157357381; 145690675; 55168340; 215499254; 29169172; 197777542; 153816650; 189353846; 154483090; 27360487; 153816533; 27359371; 10039641; 153816651; 153813776; 169191254; 77959837; 223489039; 224155181; 197766214; 197782632; 223475320; 165924309; 225111262; 50363539; 169189407; 119632772; 167630417; 147836457; 321193; 47570202; 229499565; 29565682; 5531888; 27360062; 197763227; 121592110; 227495267; 3256603; 197781048; 154500167; 154500794; 150251526; 197735635; 153813782; 167425567; 212632978; 15921449; 218151942; 163781875; 169869672; 78033426; 157354103; 40062645; 115762746; 83595848; 196018328; 115379816; 1780806; 198417694; 154500170; 108707408; 224033543; 223947683; 226443382; 237831283; 195614328; 194703406; 212274346; 149633895; 118086080; 119178984; 74007189; 197768010; 191162148; 219461701; 169213810; 167630416; 19927; 196018322; 182701819; 156341374; 115467400; 126433538; 119190145; 56422945; 50545958; 223466311; 212507121; 197762411; 169194840; 192291641; 152012802; 3831447; 67903352; 3256604; 171686416; 158294661; 154273901; 119720343. In certain embodiments, the 16s rDNA sequences set forth by NCBI gi number in this paragraph may be excluded from the claimed invention.

Alternatively or additionally, a fragment of a polynucleotide is defined as a sequence having, for example, at least 10, 20, 30, 40, 50, 75, 100, 200, 250, 500, or 1000 nucleic acids of its corresponding polynucleotide of ATCC SD 5832.

In one embodiment, a fragment of 16S rDNA sequence is defined as a contiguous sequence of the entire 16S rDNA sequence of ATCC SD-5832, wherein the fragment has at least 1000, 1050, 1100, 1150, 1200, 1250, 1300, 1350, or 1400 nucleic acids.

In another embodiment, a fragment of spoIIAB sequence is defined as a contiguous sequence of the entire spoIIAB sequence of ATCC SD-5832, wherein the fragment has at least 150, 180, 200, 220, 250, or 280 nucleic acids.

In a further embodiment, a fragment of atpA sequence is defined as a contiguous sequence of the entire atpA sequence of ATCC SD-5832, wherein the fragment has at least 600, 650, 700, 750, or 800 nucleic acids.

In yet another embodiment, a fragment of atpF sequence is defined as a contiguous sequence of the entire atpF sequence of ATCC SD-5832, wherein the fragment has at least 150, 180, 200, 220, 250, or 280 nucleic acids.

Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

Additionally or alternatively, the present invention contemplates both naturally-occurring and recombinant bacteria containing a polynucleotide that has greater than about 90%, 95%, 96%, 97%, 98%, 98.5%, 99%, or 99.5% sequence identity to the entire sequence or a fragment thereof of any of the followings sequences: 16S rDNA sequence, F1-Atpase sequence, spoIIAB sequence, an ATP synthase subunit sequence such as ATP synthase b subunit sequence, atpF sequence, or atpA sequence of ATCC SD-5832, wherein the bacteria have nematicidal activity.

Recombinant bacteria containing a polynucleotide of the present invention can be obtained by introducing polynucleotides, vectors, and expression constructs into bacterial cells by methods known in the art. Such methods include transfection, microinjection, electroporation, lipofection, cell fusion, calcium phosphate precipitation, and by biolistic methods. In one embodiment, a polynucleotide or expression construct of the invention can be introduced in vivo via a viral vector such as adeno-associated virus (AAV), herpes simplex virus (HSV), retrovirus, papillomavirus, adenovirus, and Epstein-Barr virus (EBV). Attenuated or defective forms of viral vectors that can be used with the subject invention are known in the art.

Typically, defective virus is not capable of infection after the virus is introduced into a cell. Polynucleotides, vectors, and expression constructs of the invention can also be introduced in vivo via lipofection (DNA transfection via liposomes prepared from synthetic cationic lipids) (Feigner et al., 1987). Synthetic cationic lipids (LIPOFECTIN, Invitrogen Corp., La Jolla, Calif.) can be used to prepare liposomes to encapsulate a polynucleotide, vector, or expression construct of the invention. A polynucleotide, vector, or expression construct of the invention can also be introduced in vivo as naked DNA using methods known in the art, such as transfection, microinjection, electroporation, calcium phosphate precipitation, and by biolistic methods.

The nematicidal activity of Pasteuria variants can be determined by bioassays using procedures known in the art. For instance, the nematicidal activity may be determined by applying variants to soil-dwelling nematodes at various life stages, and evaluating the effects on killing, controlling, and/or infecting nematodes; retarding the growth or reproduction of nematodes; reducing nematode population. Alternatively, the nematicidal activity may be determined by treating seeds with variants before planting, exposing the treated seeds to nematodes, and evaluating the root system, seed emergence, plant height and plant growth after planting.

Thus, a skilled artisan, benefited from the teachings of the present invention, can readily prepare and test Pasteuria variants of the present invention and determine whether the variants retain or even have improved nematicidal activity.

Large quantities of these bacteria can be produced using fermentation techniques. Sporulation occurs from the late vegetative phase of the bacteria with production of mature, dormant spores. Pasteuria endospores are not damaged by drying. Therefore, they can be stored for long periods at room temperature.

Methods for growing Pasteuria are known in the art and include, for example, the methods described in U.S. Pat. Nos. 5,094,954 and 7,067,299, both of which are incorporated herein by reference in their entirety.

The subject invention further provides bacterial endospore compositions useful for pest control. Specifically exemplified are endospore compositions of bacteria that are pathogenic to nematodes and grow in, or on, live nematode tissue.

The Pasteuria of the present invention can be delivered to seeds as unformulated spores or as a formulated liquid or solid composition, wetted powders, slurry of particles, or emulsion.

The endospores can be formulated into a wettable powder, liquid concentrate, granules or other formulations by the addition of surfactants, dispersants, inert carriers and other components to obtain a nematicidal composition that facilitates handling and application for particular target nematodes.

The commercial preparation would have a high concentration of endospores, typically in excess of 1×10⁶ spores per ml or gram, and preferably, in excess of 1×10⁹ spores per ml or gram of dry product. In general, the effective amount of spores ranges from about 1×10⁴ to 1×10¹² (or more) spores/seeds. In other embodiments, the effective amount of spores ranges from about 1×10⁵ to 1×10¹¹ spores/seeds, about 5×10⁵ to 5×10¹° spores/seeds, about 1×10⁶ to 1×10¹⁰ spores/seeds, about 5×10⁶ to 5×10⁹ spores/seeds, about 1×10⁷ to 1×10⁹ spores/seeds, or about 5×10⁷ to 5×10⁸ spores/seeds. Preferably, the spore concentration ranges from about 1×10⁶ to about 1×10⁹ spores/seed.

The composition can also include one or more of the following ingredients: other pesticides, including compounds which act only below the ground; fungicides, such as captan, thiram, metalaxyl, fludioxonil, oxadixyl, and isomers of each of those materials, and the like; herbicides, including compounds selected from carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi.

These formulation and application procedures are all well known in the art and are used with commercial strains of Pasteuria. The nematicidal composition can be sprayed or applied onto foliage to control phytopathogenic nematodes.

Another approach that can be taken is to incorporate the endospores into granules, optionally containing an attractant, and applying these granules to the soil for control of the soil-dwelling nematodes. Typically, upon contact with water the spores are released from the granule, and then the spores adhere to, and infect, nematodes. Formulated spores can also be applied as a seed-coating for root treatment or total plant treatment.

Preferably, the amount of the endospores applied is nematicidally effective. In one embodiment, less than one quart of the endospores per acre is sufficient to achieve effective nematode control.

Advantageously, the compositions are easy to apply with conventional application equipment. The endospore's mode of action makes the development of resistance unlikely. Most available nematicides must be applied to the soil before planting, because the chemicals would otherwise harm the plants. By contrast, this Pasteuria strain will not damage the plants, and can be applied at any time.

Another aspect of the invention provides seeds treated with the subject Pasteuria composition. One embodiment provides seeds having at least part of the surface area coated with the Pasteuria composition. In a specific embodiment, the Pasteuria treated seeds have a spore concentration from about 10⁶ to about 10⁹ spores per seed. The seeds may also have more spores per seed, such as, for example 1×10¹⁰, 1×10¹¹ or 1×10¹² spores per seed.

In another embodiment, the subject composition is formulated as a Pasteuria-granule mixture. The amount of Pasteuria spores to granules can range from about 1×10⁶ to about 7×10^{8 spores/g granules, about} 5×10⁶ to about 5×10⁸ spores/g granules, about 1×10⁷ to about 1×10⁸ spores/g granules, or about 3×10⁷ to about 5×10⁷ spores/g granules.

The materials and methods of the subject invention are useful for killing, controlling, and/or infecting nematodes; retarding the growth or reproduction of nematodes; reducing nematode population; and/or reducing or retarding damage to plants caused by phytopathogenic nematodes, plant-parasitic nematodes, and other soil-dwelling nematodes, including but not limited to Hoplolaimus galeatus, Meloidogyne arenaria, Pratylenchus brachyurus, Rotylenchulus reniformis, Belonolaimus longicaudatus, and Heterodera glycines. The materials and methods of the subject invention are particularly useful for killing, controlling, and/or infecting Hoplolaimus galeatus.

The materials and methods of the subject invention can be used for reducing damage to plant species, including, but not limited to, green beans, turf grasses, sweet potato, tomatoes, cotton, corn, soy beans, okra, lettuce, squash, vegetables, pineapple, tea, wheat, barley, rice, peanut, sugarcane, sorghum, tobacco, and canola.

Following is an example, which illustrates procedures for practicing the invention. This example should not be construed as limiting. All percentages are by weight and all solvent mixture proportions are by volume unless otherwise noted.

### Example 1

### Use of the Novel Pasteuria Strain for Seed Treatments

In accordance with the subject invention, Pasteuria spores can be effectively delivered to control phytopathogenic nematodes by coating the Pasteuria spores on plant seeds.

The Pasteuria spores can be coated freely onto the seeds or, preferably, they can be formulated in a liquid or solid composition before being coated onto the seeds. For example, a solid composition comprising the spores can be prepared by mixing a solid carrier with a suspension of the spores until the solid carrier is impregnated with the spore suspension. This mixture can then be dried to obtain the desired particles.

The solid carriers are preferably granules. The granules can be, for example, diatomaceous earth granules from AXIS' and/or greensgrade clay granules from PROFILE®. Various additives, such as adherents, dispersants, surfactants, and nutrient and buffer ingredients, can also be included in the carrier and spore suspension mixture.

In a specific embodiment, in addition to the spores, the coating can further comprise a layer of adherent. The adherent is preferably non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches. More examples can be found in, for example, U.S. Pat. No. 7,213,367, which is incorporated by reference herein in its entirety.

The adherent layer can help attach the spores on the surface of the seed and prevent possible drop-offs. In addition, the coating can also comprise other chemical or biological agents having a beneficial effect in combination with the Pasteuria spores for nematode control and/or for control of other pests. The coatings may also include fertilizers and other components that help promote seed germination, and/or plant growth and/or health.

Thus, the subject invention provides a method of making a Pasteuria spore coating on a plant seed. In a specific embodiment, the method comprises combining dried granule mixtures impregnated with Pasteuria spores and a seed coated with an adherent.

Although the seed treatments can be applied to a seed in any physiological state, it is preferred that the seed be in a sufficiently durable state that it incurs no damage during the treatment process. Typically, the seed has been harvested from the field; removed from the plant; and separated from any other non-seed plant material. The seed is preferably biologically stable to the extent that the treatment does not cause biological damage to the seed. In one embodiment, for example, the treatment can be applied to corn seeds that have been harvested, cleaned and dried to a moisture content below about 15% by weight. In an alternative embodiment, the seed can be one that has been dried and then primed with water and/or another material and then re-dried before or during the treatment with the Pasteuria spore composition. Within the limitations just described, the treatment can be applied to the seed at any time between harvest of the seed and sowing of the seed. As used herein, the term “unsown seed” is meant to include seed at any period between the harvest of the seed and the sowing of the seed in the ground for the purpose of germination and growth of the plant.

As used herein, when it is said that unsown seed is “treated” with the Pasteuria-containing composition, such treatment is not meant to include those practices in which Pasteuria are applied to the soil, rather than to the seed.

The Pasteuria spores are typically applied to the seeds in the form of a pesticide formulation. This formulation may contain one or more other desirable components, including but not limited to, liquid diluents, binders, fillers for protecting the seeds during stress conditions, and plasticizers to improve flexibility, adhesion and/or spreadability of the coating. In addition, it may be desirable to add to the formulation drying agents such as calcium carbonate, kaolin or bentonite clay, perlite, diatomaceous earth or any other adsorbent material. Use of such components in seed treatments is known in the art. See, e.g., U.S. Pat. No. 5,876,739. The skilled artisan, having the benefit of the current disclosure, can readily select desirable components to use in the formulation.

The seeds may also be treated with one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the seed or alternatively may be added as part of the Pasteuria composition.

Preferably, the amount of the novel composition or other ingredients used in the seed treatment should not inhibit germination of the seed, or cause phytotoxic damage to the seed.

The formulation that is used to treat the seed in the present invention can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation is preferably about 0.5% to about 99% by weight (w/w), preferably 5-40% or as otherwise formulated by those skilled in the art.

As mentioned above, other conventional inactive or inert ingredients can be incorporated into the formulation. Such inert ingredients include, but are not limited to, conventional sticking agents; dispersing agents such as methylcellulose (Methocel A15LV or Methocel A15C, for example, serve as combined dispersant/sticking agents for use in seed treatments); polyvinyl alcohol (e.g., Elvanol 51-05); lecithin (e.g., Yelkinol P), polymeric dispersants (e.g., polyvinylpyrrolidone/vinyl acetate PVPIVA S-630); thickeners (e.g., clay thickeners such as Van Gel B to improve viscosity and reduce settling of particle suspensions); emulsion stabilizers; surfactants; antifreeze compounds (e.g., urea), dyes, colorants, and the like. Further inert ingredients useful in the present invention can be found in McCutcheon's, vol. 1, “Emulsifiers and Detergents,” MC Publishing Company, Glen Rock, N. J., U.S.A., 1996. Additional inert ingredients useful in the present invention can be found in McCutcheon's, vol. 2, “Functional Materials,” MC Publishing Company, Glen Rock, N.J., U.S.A., 1996.

The coating formulations of the present invention can be applied to seeds by a variety of methods, including, but not limited to, mixing in a container (e.g., a bottle or bag), mechanical application, tumbling, spraying, and immersion. A variety of active or inert material can be used for contacting seeds with pesticides according to the present invention, such as conventional film-coating materials, including but not limited to, water-based film coating materials such as SEPIRET™ (Seppic, Inc., Fairfield, N.J.) and OPACOAT™ (Berwind Pharm. Services, Westpoint, Pa.).

Seed coating methods and compositions that are known in the art are useful when they are modified by the addition of one of the embodiments of the present invention. Such coating methods and apparatus for their application are disclosed in, for example, U.S. Pat. Nos. 5,918,413, 5,891,246, 5,554,445, 5,389,399, 5,107,787, 5,080,925, 4,759,945 and 4,465,017. Seed coating compositions are disclosed, for example, in U.S. Pat. Nos. 5,939,356, 5,882,713, 5,876,739, 5,849,320, 5,834,447, 5,791,084, 5,661,103, 5,622,003, 5,580,544, 5,328,942, 5,300,127, 4,735,015, 4,634,587, 4,383,391, 4,372,080, 4,339,456, 4,272,417 and 4,245,432, among others.

Binders that are useful in the present invention preferably comprise an adhesive polymer that may be natural or synthetic and is preferably without substantial phytotoxic effect on the seed to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

The amount of Pasteuria that is used for the treatment of the seed will vary depending upon the type of seed and the type of active ingredients, but the treatment will comprise contacting the seeds with an amount of the Pasteuria that is pesticidally effective. As used herein, a nematicidally effective amount means that amount of Pasteuria that will kill the nematodes, or will consistently reduce or retard the amount of damage caused by nematodes.

The pesticides that are used in the treatment must not inhibit germination of the seed and should be efficacious in protecting the seed and/or the plant during that time in the nematode's life cycle in which it causes injury to the seed or plant. In general, the coating will be efficacious for approximately 1 hour to 120 days after sowing.

The coatings formed with the pesticide are preferably of the type that are capable of effecting a slow rate of release of the pesticide by diffusion or movement through the matrix to the surrounding medium.

In addition to the coating layer, the seed may be treated with one or more of the following ingredients: other pesticides including fungicides and herbicides; herbicidal safeners; fertilizers and/or biocontrol agents. These ingredients may be added as a separate layer or alternatively may be added in the pesticidal coating layer.

The pesticide formulation may be applied to the seeds using a variety of techniques and machines, such as fluidized bed techniques, the roller mill method, rotostatic seed treaters, and drum coaters. Other methods, such as spouted beds may also be useful. The seeds may be presized before coating. After coating, the seeds are typically dried and then transferred to a sizing machine for sizing. Such procedures are known in the art.

The Pasteuria treated seeds may also be enveloped with a film overcoating to protect the coating. Such overcoatings are known in the art and may be applied using fluidized bed and drum film coating techniques.

In another embodiment of the present invention, the Pasteuria spores can be introduced onto a seed by use of solid matrix priming. For example, a quantity of the Pasteuria spores can be mixed with a solid matrix material and then the seed can be placed into contact with the solid matrix material for a period to allow the pesticide to be introduced to the seed. The seed can then optionally be separated from the solid matrix material and stored or used, or the mixture of solid matrix material plus seed can be stored or planted directly. Solid matrix materials which are useful in the present invention include polyacrylamide, starch, clay, silica, alumina, soil, sand, polyurea, polyacrylate, or any other material capable of absorbing or adsorbing the pesticide for a time and releasing that pesticide into or onto the seed. It is useful to make sure that the pesticide and the solid matrix material are compatible with each other. For example, the solid matrix material should be chosen so that it can release the pesticide at a reasonable rate, for example over a period of minutes, hours, or days.

Unlike the vegetative form of the bacteria, Pasteuria spores are not damaged by drying and they can be stored for long periods at room temperature. Therefore, one advantage of the subject invention is that the drying and other harsh steps used in coating methods can be applied to the subject invention for seed coating without significantly reducing the effectiveness of the spores. The long shelf life of seeds of the subject invention also allows variations in planting schedules. In addition, the survival rate of the Pasteuria spores is much higher than the vegetative form of the bacteria during transport and sowing once placed in the soil.

In general, the effective amount of spores ranges from about 1×10⁵ to 1×10¹² (or more) spores/seed. Preferably, the spore concentration is about 1×10⁶ to about 1×10⁹ spores/seed.

In one embodiment, to obtain the granule mixtures, the ratio of Pasteuria spores to granule is about 3×10⁷ to 5×10⁷ spores/g granules. In a specific embodiment, about 3-5 ml of a Pasteuria spore suspension containing about 2×10⁷ spores/ml of buffer is added to about 2 g of granules. The ratio can depend on the granule types. For example, about 5 ml of spore suspension can be applied to 2 g of AXIS® granules while about 3 ml of spore suspension is preferred for the same amount of PROFILE® granules. The adherent can be any commercial glue biocompatible with the seed and soil, such as ELMER′S clear school glue containing polyvinyl acetate.

An extra heat-treatment step can be included in order to kill nematodes if the spores are produced in nematode hosts. The heat-treatment step can be applied to the spore suspension before mixing with granules. Alternatively, the step can be applied after formation of granule mixtures.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. An isolated Pasteuria strain that is deposited as ATCC accession number SD-5832, or a nematicidally active mutant or variant of said deposited strain.

2. The isolated Pasteuria strain, according to claim 1, wherein the strain is a variant of said deposited strain, wherein said variant has a polynucleotide sequence that is at least 95% identical to a sequence selected from the group consisting of 16S rDNA sequence, F1-Atpase sequence, spoIIAB sequence, ATP synthase b subunit sequence, atpF sequence, and atpA sequence of said deposited strain, and fragments thereof.

3. The isolated Pasteuria strain, according to claim 1, wherein said variant has a polynucleotide sequence that is at least 98% identical to 16S rDNA sequence of said deposited strain, or a fragment thereof.

4. The isolated Pasteuria strain, according to claim 1, which is deposited as ATCC accession number SD-5832.

5. The isolated Pasteuria strain, according to claim 1, which is active against Hoplolaimus galeatus.

6. A nematicidal composition comprising a nematicidally effective amount of a Pasteuria strain of claim 1, and an agricultural carrier.

7. The composition, according to claim 6, wherein the carrier is a seed treatment.

8. A method for controlling nematodes, wherein said method comprises contacting nematodes with a nematicidally effective amount of a Pasteuria strain of claim 1.

9. The method, according to claim 8, wherein the nematode is Hoplolaimus galeatus.

10. The method, according to claim 8, used to protect a crop selected from the group consisting of green beans, turf grasses, sweet potato, tomatoes, cotton, corn, soy beans, okra, lettuce, squash, vegetables, pineapple, tea, wheat, barley, rice, peanut, sugarcane, sorghum, tobacco, and canola.

11. The method, according to claim 10, wherein the crop is turf grasses.

12. The method, according to claim 8, wherein the Pasteuria strain is applied to soil.
